(19) United States
(12) Patent Application Publication
(10) Pub. No.: US 2022/0177513 A1
(43) Pub. Date: Jun. 9, 2022

(54) Process for the Preparation of Abaloparatide

(71) Applicant: Mylan Laboratories Limited, Hyderabad (IN)

(72) Inventors: Ananda Kuppanna, Hyderabad (IN); Sureshbabu Jayachandra, Hyderabad (IN); Anil Kumar Tripathi, Hyderabad (IN); Bulliraju Kamana, Hyderabad (IN); Chandrashekhar Shanigaram, Hyderabad (IN)

(21) Appl. No.: 17/598,957

(22) PCT Filed: Mar. 24, 2020
(86) PCT No.: PCT/IN2020/050277

§ 371 Date: Sep. 28, 2021

(30) Foreign Application Priority Data
Mar. 29, 2019 (IN) 201941012325

(51) Int. Cl. C07K 1/10; C07K 14/635

(52) U.S. Cl. CPC C07K 1/10; C07K 14/635

(57) ABSTRACT

A process for the preparation of Abaloparatide or pharmaceutically acceptable salts. Also described is pure Abaloparatide or pharmaceutically acceptable salts thereof obtained from the process for the preparation of Abaloparatide.

### CROSS-REFERENCE TO RELATED APPLICATIONS

This application is the United States national phase of International Application No. PCT/IN2020/050277 filed Mar. 24, 2020, and claims the benefit of the filing date of Indian Provisional Application No. 201941012325 filed on Mar. 29, 2019, the contents of which are incorporated herein in their entirety by reference.

### FIELD

The present invention relates to an improved process for the preparation of Abaloparatide or pharmaceutically acceptable salts.

### BACKGROUND

Abaloparatide is used for the treatment of postmenopausal women with osteoporosis under the brand name of TYMLOS, marketed by Radius Health Inc. TYMLOS injection for subcutaneous administration contains abaloparatide, a synthetic 34 amino acid peptide. Abaloparatide is an analog of human parathyroid hormone related peptide, PTHrP(1-34). It has 41% homology to hPTH(1-34) (human parathyroid hormone 1-34) and 76% homology to hPTHrP(1-34) (human parathyroid hormone-related peptide 1-34).

Abaloparatide has a molecular formula of C₁₇₄H₃₀₀N56O₄₉ and a molecular weight of 3961 daltons with the amino acid sequence shown below:

Ala-Val-Ser-Glu-His-Gln-Leu-Leu-His-Asp-Lys-Gly-
Lys-Ser-Ile-Gln-Asp-Leu-Arg-Arg-Arg-Glu-Leu-Leu-
Glu-Lys-Leu-Leu-Aib-Lys-Leu-His-Thr-Ala-NH2.

Abaloparatide is disclosed in U.S. Pat. No. 5,969,095 B2, which is hereby incorporated by reference.

There is no sufficient data available in the literature for the preparation of Abaloparatide such as loading capacity of the resin. Therefore, there exists a need to develop an alternate and improved process for the preparation of Abaloparatide with improved yield and purity. Further the process is simple, convenient and cost-effective for large scale production.

### SUMMARY

The present invention relates to an improved process for the preparation of Abaloparatide.

The main aspect of the present invention is to provide an improved process for the preparation of Abaloparatide by solid phase peptide synthesis, followed by purification to get Abaloparatide. The flow chart description of the process is as shown in scheme-1

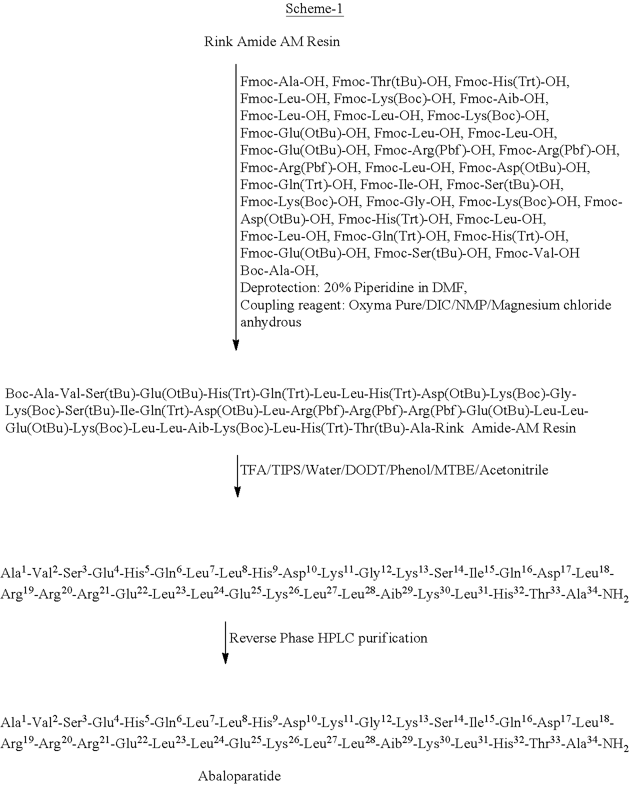

### DETAILED DESCRIPTION

The main object of the present invention is to provide an improved process for the preparation of Abaloparatide.

For the purpose of clarity and as an aid in the understanding of the invention, as disclosed and claimed herein, the following terms and abbreviations are defined below

### ABBREVIATIONS

Boc Di-tert-butyl-dicarbonate
tBu tert-butyl
DCM dichloromethane
DMF N,N′-Dimethylformamide
DIEA Diisopropylethylamine
DIC N,N-diisopropylcarbodiimide
DODT 2,2′-(ethylenedioxy) diethanethiol
Fmoc 9-fluorenylmethoxycarbonyl
HOBt N-hydroxybenzotriazole
HBTU O-Benzotriazole-N,N,N′,N′-tetramethyl-uronium-hexafluoro-phosphate
MTBE Methyl tert-butyl ether
NMP N-Methyl-2-pyrrolidone
DVB Divinylbenzene
Lys Lysine
Thr Threonine
TFA trifluoroacetic acid
TIPS triisopropylsilane
His Histidine
Ala Alanine
Glu Glutamic acid
Gly Glycine
Ser Serine
Asp Aspartic acid
Val Valine
Tyr Tyrosine
Aib 2-Aminoisobutyric acid
Leu Leucine
Gln Glutamine
IIe Isoleucine
Arg Arginine
Pbf pentmethyldihydrobenzofuransulfonyl
Trt Trityl
Oxyma Pure Ethyl cyano(hydroxyimino)acetate

In yet another embodiment of the present invention is to provide a process for the preparation of Abaloparatide comprising the steps of:
a) anchoring first protected terminal amino acid to a resin;
b) capping the resin obtained in step a);
c) selectively deprotecting the amino group;
d) coupling carboxyl terminus of the next N-protected amino acid to the amine group in presence of a coupling reagent;
e) repeating steps c) and d) to form a thirty-four peptide sequence;
f) cleaving the peptide with cocktail mixture from the resin to isolate straight chain of crude Abaloparatide; and
g) purifying by reverse phase HPLC to isolate pure Abaloparatide.

According to the present invention, the resin used for synthesis of peptide undergoes swelling in presence of a solvent selected from dichloromethane and N, N-dimethylformamide. The resin used is selected from Rink-Amide resin.

The swelled resin is treated with 20% piperidine in DMF to get N-terminal free Rink-Amide-AM resin, subsequently protected amino acid washed in presence of anhydrous magnesium chloride, NMP, Oxyma Pure, DIC, HOBT, HBTU and DIPEA for a desired period of time to form peptide bond. The solvent used is selected from dichloromethane, tetrahydrofuran, N,N-dimethylformamide, N,N-dimethylacetamide, N-methyl-2-pyrrrolidone or mixtures thereof.

Before proceeding to the next step, the unreacted linkers on the resin (polymer) are protected (capped) to avoid the undesired peptide chain formation. The reagent used for the capping is acetic anhydride, pyridine and dichloromethane.

According to the present invention, the deprotection of the amino acid attached to the resin is done selectively in the presence of a nucleophilic base such as 20% piperidine in presence of a solvent. The solvent used is selected from N, N-dimethylformamide, methylene chloride, tetrahydrofuran, N-methyl pyrrolidine or mixture thereof.

According to the present invention, selectively deprotected the amino group is coupled with next N-protected amino acid in a solvent in presence of a coupling reagent. The solvent used for the coupling reaction is selected from dichloromethane, tetrahydrofuran, dimethylformamide, N-methylpyrolidone or mixture thereof. The coupling agent used for the coupling of the amino acids is selected from Oxyma pure, HBTU, HOBt and DIEA.

According to the present invention, the resin cleavage and global deprotection (a process for deprotecting the protected amino acid in the peptide, which has additional functional groups) of the peptide is carried out with a cocktail mixture. The cleavage of the peptide from the resin involves treating the protected peptide anchored to the resin with an acid having at least one scavenger. The acid utilized in the cleavage reagent is TFA. The amount of TFA used for the purpose of cleavage of peptide from the resin and global deprotection in the cocktail mixture may range from 80-90%. The scavengers used are selected from TIPS, phenol, thioanisole, water, DODT or mixtures thereof. The particular cocktail mixture used for the cleavage of the peptide from resin is 90% TFA, 5% water, 5% TIPS and DODT.

The two cocktail mixture for the cleavage of the peptide from resin are disclosed in the prior art and they are.

a) TFA/EDT/Thioanisole/DCM/TIPS in the ratio of about 80%/5%/5%/5%/5% or 80%/5%/3.5%/3.5%/8% respectively and

b) TFA/phenol/Thioanisole/DCM/TIPS/H20 in the preferred volumes of 80%/5%/3.33%/3.33%/5%/3.33% respectively or 80%/5%/3.5%/3.5%/8% respectively.

However, it does not disclose the use of the above conditions for the preparation of abaloparatide.

According to the present invention, the resin after the completion of the reaction is optionally washed with solvents such as DMF and DCM to remove residual reagents and byproducts. The process is repeated if desired and before proceeding to next step.

According to present invention, the isolation of Abaloparatide is carried out by precipitating with ether solvent to get Abaloparatide as a solid. Ether solvents that are used for precipitation is selected from methyl tert-butyl ether, diethyl ether, t-butyl methyl ether, isopropyl ether or mixtures thereof.

The process for the preparation of abaloparatide is summarized in synthetic scheme-1 depicted below.

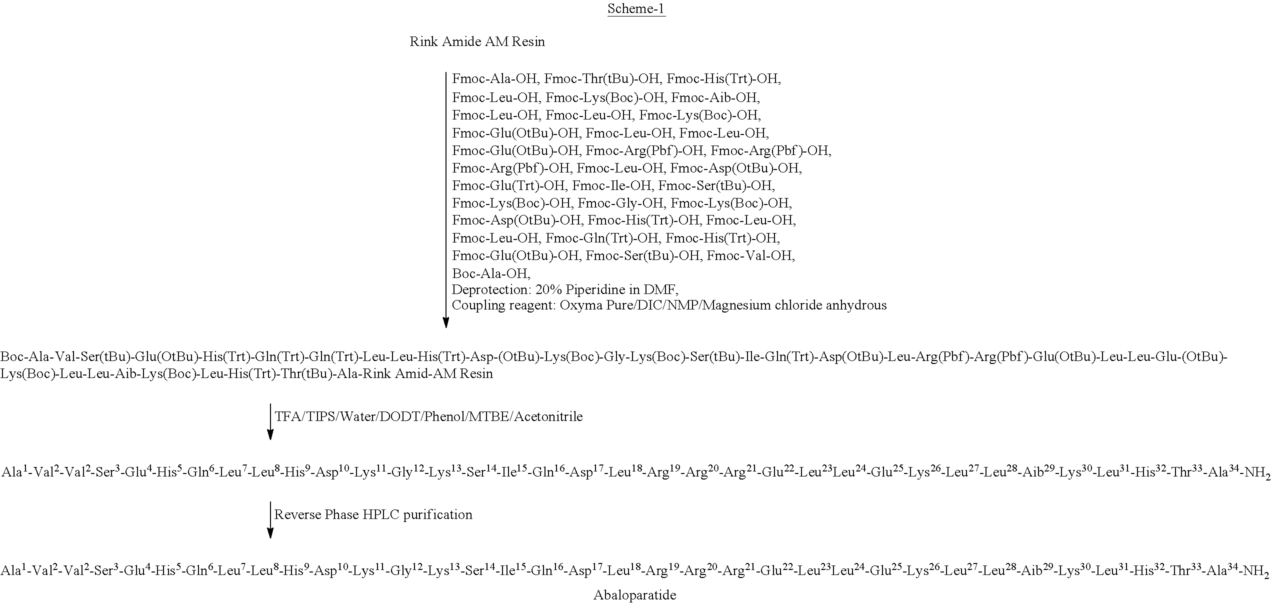

According to the present invention, solid phase of peptide synthesis for preparation of protected Abaloparatide-Rink Amide AM resin, the loading of first protected amino acid (Fmoc-Ala-OH) to Rink amide resin plays a significant role. It has been observed that when the solid phase peptide synthesis reaction has carried out loading of first protected amino acid with matrix about 0.50 to 0.6 mmol/g, then the crude purity of Abaloparatide was about 50% only. However, when loading of first protected amino acid with matrix about 0.35 to 0.45 mmol/g was used, the crude purity was obtained about 70-73%. The results are summarized in the following table.

The Rink amide AM resin used for the peptide synthesis and their loading capacity are listed below.

| Resin | Matrix | Loading | Purity |
|---|---|---|---|
| Rink amide AM resin | Amino methyl polystyrene | 0.52 mmol/g | 53.0 |
| | crosslinked with 1% DVB | | |
| Rink amide AM resin | Amino methyl polystyrene | 0.40 mmol/g | 71.0 |
| | crosslinked with 1% DVB | | |
| Rink amide AM resin | Amino methyl polystyrene | 0.40 mmol/g | 73.0 |
| | crosslinked with 1% DVB | | |

The resin used for the preparation of Abaloparatide as support material is Rink amide AM resin (0.40 mmol/g). The selection of polymeric support and attached linker is very critical for overall outcome of the solid phase peptide synthesis. Rink amide AM resin is found to be very effective for the preparation of Abaloparatide and are comprising of grafted copolymers consisting of a low cross-linked polystyrene, but the loading capacity is 0.40 mmol/g.

The Rink amide AM resin is used in the process of Abaloparatide in low load in 0.40 mmol/g with amino methyl polystyrene type linker has advantages over with high loading in 0.52 mmol/g in achieving the better purity. The advantage of using low load resin is that significantly more peptide for unit measure of beads could be produced. This is a consequence of the fact that higher concentrations of reagents and reactants can be achieved with low load resins. Smaller vessel sizes could be employed to generate a given amount of peptide and at least 50% less wash solvents needed while using low loaded resins. For the scale up of the solid phase attractive it is important to reduce the large amounts of reagents typically employed in solid phase peptide synthesis.

Yet another embodiment of the present invention is to provide a process for the preparation of Abaloparatide with reduced level of below listed impurities.

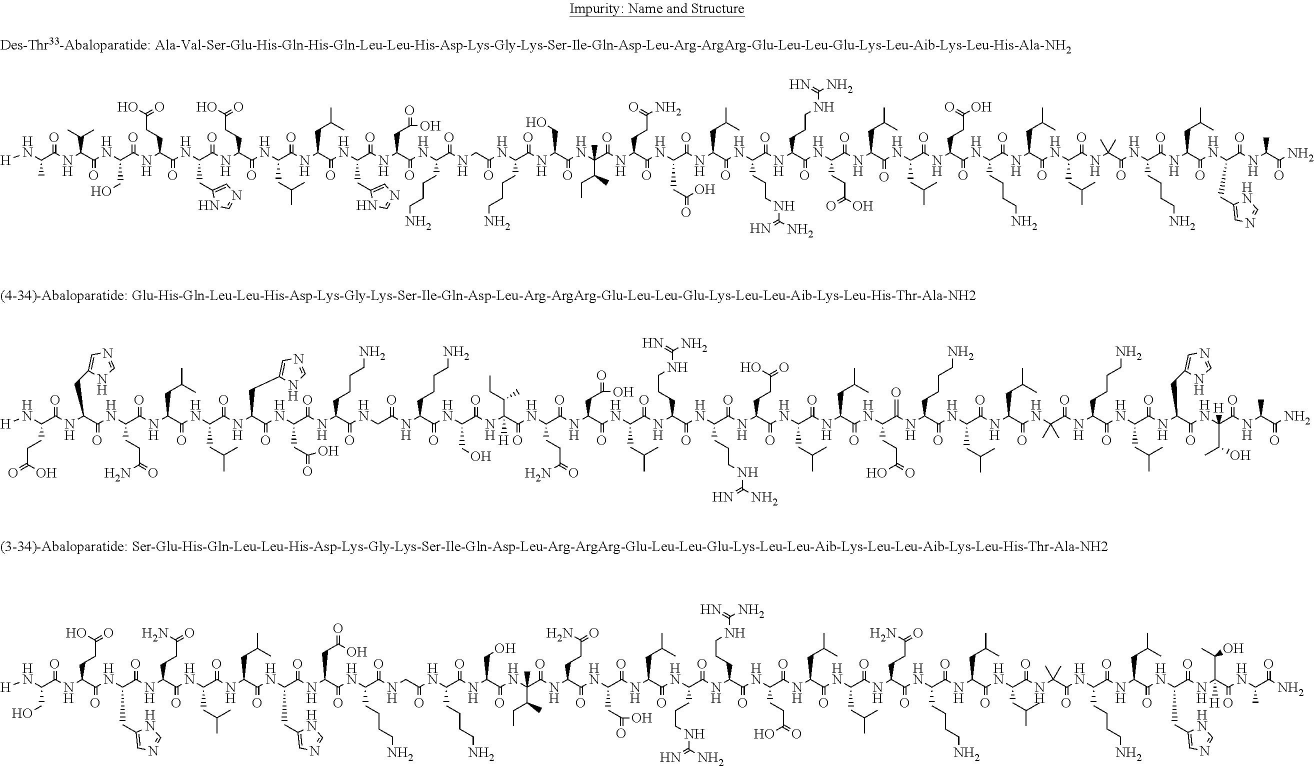

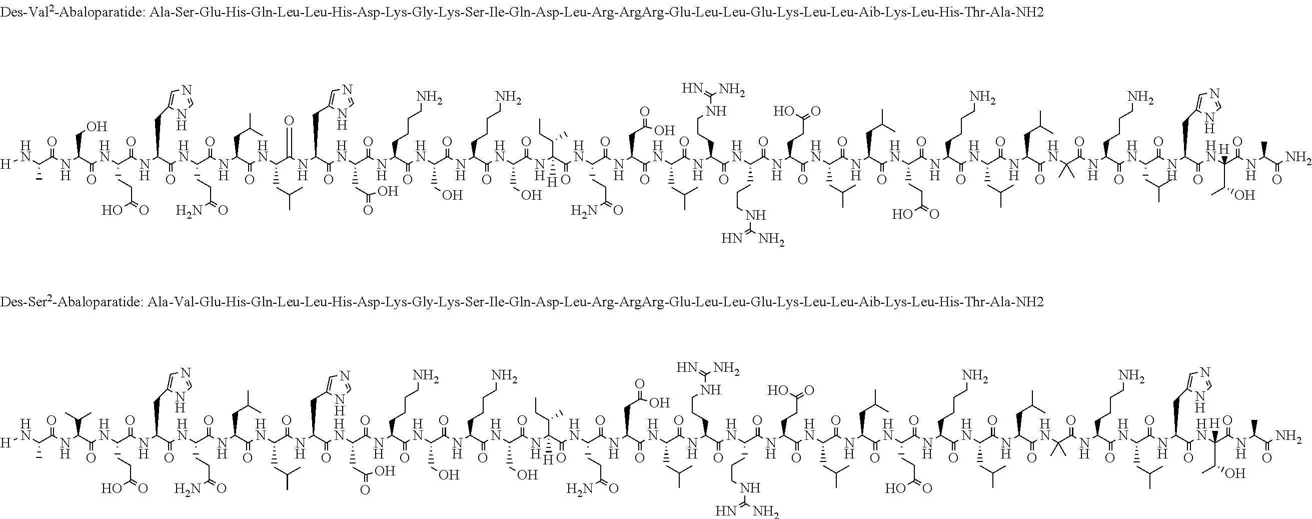

Yet another embodiment of the present invention is to provide a process for the preparation of Abaloparatide with HPLC purity of at least 99.0%.

Yet another embodiment of the present invention is to provide, Abaloparatide containing low levels of impurities selected from des-Thr³³-Abaloparatide, (4-34)-Abaloparatide, (3-34)-Abaloparatide, des-Val²-Abaloparatide and Des-Ser³-Abaloparatide.

Certain embodiment of the present invention is to provide Abaloparatide contains less than 0.10% Des-Thr³³-Abaloparatide impurity.

Certain embodiment of the present invention is to provide Abaloparatide contains less than 0.20% (4-34)-Abaloparatide impurity.

Certain embodiment of the present invention is to provide Abaloparatide contains less than 0.10% (3-34)-Abaloparatide impurity.

Certain embodiment of the present invention is to provide Abaloparatide contains less than 0.10% Des-Vale²-Abaloparatide impurity.

Certain embodiment of the present invention is to provide Abaloparatide contains less than 0.10% Des-Ser³-Abaloparatide impurity.

Certain embodiment of the present invention is to provide a pharmaceutical composition comprising Abaloparatide and pharmaceutically acceptable carrier.

The following example is provided to illustrate the process of the present invention. However, they are not intended to limit the scope of an invention.

### EXAMPLES

### Example-1: Preparation of Protected Abaloparatide-Rink Amide Resin

In a clean dry peptide synthesizer vessel charged 100 g of Rink-Amide AM Resin and 1000 ml of DCM. Allowed the resin to swell for 1 hr. Drain the solvent. Deprotect the Fmoc group by using 20% piperidine (2×1000 ml) followed by washing the resin with 3×1000 ml of DMF and 1×1000 ml of DCM. Dissolve the 1.5 eq. of Fmoc-Ala-OH, 1.5 eq. of 1-Hydroxybenzotriazole (HOBT) anhydrous in 1000 ml of N-Methyl-2-pyrrolidone (NMP) at temp. 20-25° C. and added 2.0 eq. of N,N-diisopropyl carbodiimide (DIC). Charged the above solution into peptide synthesizer and stirred the reaction mass for 2 to 3 hrs. at temp. 20-25° C. Drain the solvent and resin was washed with 3×1000 ml of DMF followed by 1×1000 ml of DCM. Checked the loading of first amino acid. (Limit: 0.3 to 0.7 mmol/g). To end cap the resin, prepared a mixture of 880 ml of (DCM), 60 ml of acetic anhydride and 60 ml of pyridine and charged the above solution into peptide synthesizer vessel. Stirred the mass for 1 hr. Drain the solvent and washed the resin with 3×1000 ml of DMF followed by 1×1000 ml of DCM. Deprotect the Fmoc group by using 20% piperidine in DMF (2×1000 ml) followed by washing the resin with 3×1000 ml of DMF and 1×1000 ml of DCM. The Fmoc deprotection was monitored by using Kaiser Test. Continue the coupling by dissolving the 2.0 eq. of Fmoc-Thr(tBu)-OH, 2.0 eq. of 1-Hydroxybenzotriazole (HOBT) anhydrous, 2.0 eq. N, N, N′, N′-Tetramethyl-O-(1H-benzotriazol-1-yl) uronium hexafluorophosphate (HBTU) in 1000 ml of N-Methyl-2-pyrrolidone (NMP) at temp. 20-25° C. to get a clear solution followed by addition of 2.0 eq. of N,N-diisopropyl ethyl amine (DIPEA). Charged the above solution into peptide synthesizer vessel and stirred the reaction mass for 2 to 3 hrs. at temp. 20-25° C. The progress of reaction was monitored by using Kaiser test. After completion of reaction, drain the solvent and resin was washed with 1000 ml of DMF. The Fmoc was deprotected by using 20% piperidine in DMF (2×1000 ml) followed by washing the resin with 3×1000 ml of DMF and 1×1000 ml of DCM. After deprotection of Fmoc group, the coupling sequence of subsequent amino acids, i.e. Fmoc-Thr(tBu)-OH (2.0 eq.), Fmoc-His(Trt)-OH (2.0 eq.), Fmoc-Leu-OH (2.0 eq.), Fmoc-Lys(Boc)-OH (2.0 eq.), Fmoc-Aib-OH (4eq.), Fmoc-Leu-OH (2.0 eq.), Fmoc-Leu-OH (2.0 eq.), Fmoc-Lys(Boc)-OH (2.0 eq.), Fmoc-Glu(OtBu)-OH(2.0 eq.), Fmoc-Leu-OH (2.0 eq.), Fmoc-Leu-OH (2.0 eq.), Fmoc-Glu(OtBu)-OH (2.0 eq.), Fmoc-Arg(Pbf)-OH (2.0 eq.), Fmoc- Arg(Pbf)-OH (2.0 eq.), Fmoc-Arg(Pbf)-OH (2.0 eq.), Fmoc-Leu-OH (2.0 eq.), Fmoc-Asp(OtBu)-OH (2.0 eq.), Fmoc-Gln(Trt)-OH (2.0 eq.), Fmoc-IIe-OH (2.0 eq.), Fmoc-Ser(tBu)-OH (3.0 eq.), Fmoc-Lys(Boc)-OH (3.0 eq.), Fmoc-Gly-OH (3.0 eq.), Fmoc-Lys(Boc)-OH (3.0 eq.), Fmoc-Asp(OtBu)-OH (3.0 eq.), Fmoc-His(Trt)-OH (3.0 eq.), Fmoc-Leu-OH (3.0 eq.), Fmoc-Leu-OH (3.0 eq.), Fmoc-Gln(Trt)-OH (3.0 eq.), Fmoc-His(Trt)-OH (3.0 eq.), Fmoc-Glu(OtBu)-OH (3.0 eq.), Fmoc-Ser(tBu)-OH (3.0 eq.), Fmoc-Val-OH (3.0 eq.) and Boc-Ala-OH(3.0 eq.) were performed. The coupling reagent was used as 1-Hydroxybenzotriazole (HOBT) anhydrous, N, N, N′, N′-Tetramethyl-O-(1H-benzotriazol-1-yl) uronium hexafluorophosphate (HBTU), N,N-diisopropyl ethyl amine (DIPEA) as base and N-Methyl-2-pyrrolidone (NMP) as a solvent. The deprotection of Fmoc was carried out using 20% piperidine in DMF as a solvent. After completion of synthesis washed the resin with methanol (2×1000 ml) followed by MTBE (2×1000 ml). Finally, the resin containing the peptide was dried under vacuum tray dryer for 2 hrs., temp.40-45° C. Wt. of Protected Abaloparatide Resin: 300 g Molar yield: 82.6%

### Example 2: Preparation of Crude Abaloparatide

In a round bottom flask charged 800 ml of trifluoroacetic acid (TFA), 80 ml of triisopropylsilane (TIPS), 80 ml of 2,2′-(ethylenedioxy) diethanethiol (DODT) and 50 ml of water. Cooled the mixture to 0 to 10° C. Slowly added the Protected Abaloparatide 100 g over a period for 5 min. Stir the reaction mass for 30 min at temp. 0 to 10° C. Raised the temperature of reaction mass to 20-25° C. and stirred the reaction mass for 3 hrs. Filter the reaction mass and keep the mother liquor aside. In another round bottom flask charged 3000 ml of MTBE and cooled the mass to 0 to −10° C. Slowly added the TFA solution (mother liquor) into cooled MTBE solution over a period of 30 min. at temp. 0 to −10 ° C. The product was precipitate out. Maintain the temperature of reaction mass 0 to −10° C. for 30 min. Filtered the reaction mass and washed the cake with 3×1000 ml MTBE. Charged the wet cake into flask and added 600 ml of ethyl acetate and 30 ml of acetic acid. Stirred the mass for 12 hrs. The product was isolated by filtration and washed the cake with 100 ml of ethyl acetate. Dry the product under reduced pressure at temperature 40-45° C. Dry weight of Crude Abaloparatide: 36 g

Threotical yield: 39 g

Molar yield: 78.0%

Purity: 70%

Impurity content

Des-Thr³³-Abaloparatide:0.60%.

(4-34)-Abaloparatide:1.2%.

(3-34)-Abaloparatide:1.1%.

Des-Val²-Abaloparatide:1.1%.

Des-Ser³-Abaloparatide:1.2%.

### Example-3: Preparation of Protected Abaloparatide-Rink Amide Resin

In a clean dry peptide synthesizer vessel charged 250 g of Rink-Amide AM Resin (vendor loading 0.3 to 0.7 mmol/g) and 1000 ml of DCM. Allowed the resin to swell for 1 hr. Filter the solvent. Deprotect the Fmoc group by using mixture of 20% piperidine (2×1000 ml) at temp. 25±5° C. followed by washing the resin with 3×1000 ml of DMF and 1×1000 ml of DCM. Mix 31.1 g (0.4 eq.) of Fmoc-Ala-OH, 21.3 g (1.5 eq.) of oxyma pure, 78 ml (5.0 eq.) of DIC (isopropyl carbodiimide) in 1000 ml of N-Methyl-2-pyrrolidone (NMP) at temp. 20-25° C. under nitrogen atmosphere. Charged the above solution into peptide synthesizer and stirred the reaction mass for 4 to 5 hrs. at temp. 20-25° C. Filter the mass and washed the resin with 3×1000 ml of DMF followed by 1×1000 ml of DCM. The loading of first amino acid was 0.4 mmol/g (Limit: 0.3 to 0.5 mmol/g). Caped the active site of Rink Amide AM resin using a mixture of 1000 ml of (DCM), 60 ml of acetic anhydride and 60 ml of pyridine at temp. 25±5° C. Charged the above solution into peptide synthesizer vessel. Stirred the mass for 1 hr. at temp. 20-25° C. Filter the solvent and repeat the capping operation twice. After filtration of solvent, washed the resin with 3×1000 ml of DMF followed by 1×1000 ml of DCM. Check the Kaiser test (Colorless of beads). Deprotect the Fmoc group by using mixture of 20% piperidine (2×1000 ml) and 1% (w/v) of HOBT at temp. 25±5° C. followed by washing the resin with 3×1000 ml of DMF and 1×1000 ml of DCM. The Fmoc deprotection was monitored by Kaiser Test. Continue the coupling with subsequent protected amino acids (typically 2 to 6 mol. eq.), coupling reagent Oxyma Pure /DIC typically 2 to 6 eq., additive 250 mg of anhydrous magnesium chloride and NMP was used as a solvent. When the coupling of protected amino acid is not complete, repeated the coupling reaction. The reaction was monitored by Kaiser test. Still if reaction is not complete, capped the unreacted site of peptide using acetic anhydride/pyridine and DCM was used as a solvent. The coupling reaction of subsequent amino acids, i.e. Fmoc-Thr(tBu)-OH, Fmoc-His(Trt)-OH, Fmoc-Leu-OH, Fmoc-Lys (Boc)-OH, Fmoc-Aib-OH, Fmoc-Leu-OH, Fmoc-Leu-OH (OH, Fmoc-Lys(Boc)-OH, Fmoc-Glu(OtBu)-OH, Fmoc-Leu-OH, Fmoc-Leu-OH, Fmoc-Glu(OtBu)-OH, Fmoc-Arg(Pbf)-OH, Fmoc-Arg(Pbf)-OH, Fmoc-Arg(Pbf)-OH, Fmoc-Leu-OH, Fmoc-Asp(OtBu)-OH, Fmoc-Gln (Trt)-OH, Fmoc-IIe-OH, Fmoc-Ser(tBu)-OH, Fmoc-Lys(Boc)-OH, Fmoc-Gly-OH, Fmoc-Lys(Boc)-OH, Fmoc-Asp(OtBu)-OH, Fmoc-His(Trt)-OH, Fmoc-Leu-OH, Fmoc-Leu-OH, Fmoc-Gln(Trt)-OH, Fmoc-His(Trt)-OH, Fmoc-Glu(OtBu)-OH, Fmoc-Ser(tBu)-OH, Fmoc-Val-OH and Boc-Ala-OH were performed. Typically coupling reaction has been performed at temperature 36-40° C. For Fmoc-His (Trt)-OH the coupling reaction has been performed at ambient temperature. The Fmoc was deprotected by stirring the resin with mixture of 20% piperidine in DMF (2×1000 ml) and 1% (w/v) of HOBT for 15 min. at temp. 25±5° C. followed by washing the resin with 3×1000 ml of DMF and 1×1000 ml of DCM. After completion of synthesis washed the resin with DMF (2×1000 ml) and 1000 ml of DCM followed by MTBE (2×1000 ml). Finally, the resin containing the peptide was dried under vacuum tray dryer for 3 to 4 hrs., temp. not more than 40-45° C. Wt. of Protected Abaloparatide Resin: 780 g

Molar yield: 86.6%

### Example-4: Preparation of Crude Abaloparatide

In a round bottom flask charged 800 ml of trifluoroacetic acid (TFA), 80 ml of triisopropylsilane (TIPS), 50 ml of phenol, 80 ml of 2,2′-(ethylenedioxy) diethanethiol (DODT) and 50 ml of water. Cooled the mixture to 0 to 10° C. Slowly added the Protected Abaloparatide 100 g over a period for 5 min. at 0 to 10° C. Stir the reaction mass for 30 min at temp. 0 to 10° C. Raised the temperature of reaction mass to 20-25° C. and stirred the reaction mass for 3 to 4 hrs. Filter the reaction mass and keep the mother liquor aside. In another round bottom flask charged 3000 ml of MTBE and cooled the mass −10 to −20° C. Slowly added the TFA solution (mother liquor) into cooled MTBE solution over a period of 30 min. at temp. 0 to −10° C. The product was precipitate out. Maintain the temperature of reaction mass −0 to −10° C. for 30 min. Filtered the reaction mass and washed the cake with 3×1000 ml of MTBE. Charged the wet cake into flask and charge 600 ml of acetonitrile. Stirred the mass for about 2 hrs. The product was isolated by filtration and washed the cake with 500 ml of MTBE. Dry the product under reduced pressure at temperature 25-35° C.

Weight of crude Abaloparatide: 39 g

Molar yield: 85.2%

Purity: 70%

Impurity content:

Des-Thr³³-Abaloparatide:0.50%.

(4-34)-Abaloparatide:1.0%.

(3-34)-Abaloparatide:1.0%.

Des-Val²-Abaloparatide:1.0%.

Des-Ser³-Abaloparatide:1.0%.

### Example-5: Purification of Abaloparatide

The purification process is accomplished by preparative HPLC using reverse phase C18 material as the support. Preparation of the column (equilibration) is accomplished by washing with 0.1% aqueous TFA to avoid elution of the product in the loading phase. The peptide is loaded onto the column and eluted with mobile phase gradient buffers and the organic modifier acetonitrile. The fractions are monitored by analytical UPLC as they elute from the column. The purification process for Abaloparatide is divided into three stages. The first stage is purification of the crude peptide using gradient elution buffers consisting of 0.1% TFA and acetonitrile. The second stage is purification of the first stage fractions with peptide purity ≥97% (main pool) using 0.10% acetic acid and acetonitrile. Third stage is salt exchange using 0.1 M ammonium acetate (NH₄OAc) buffer and gradient elution buffers consisting of 0.05% acetic acid and acetonitrile while simultaneously converting the peptide into the acetate salt form. Upon completion of the final purification stage, fractions that meet the purity criteria of ≥98.0% are lyophilized and the yield of the purified peptide is determined and recorded. By using 100 g of crude Abaloparatide lOg of purified Abaloparatide was obtained.

Yield: 10 g

Molar yield: 10.0%

Purity: 99.5%

Impurity content:

Des-Thr³³-Abaloparatide:0.10%

(4-34)-Abaloparatide:0.20%

(3-34)-Abaloparatide:0.10%

Des-Val²-Abaloparatide:0.10%.

Des-Ser³-Abaloparatide:0.10%.

1. A process for the preparation of Abaloparatide comprising the steps of:
a) anchoring a first protected terminal amino acid to a resin,
b) capping the resin obtained in step a),
c) selectively deprotecting the amino group,
d) coupling a carboxyl terminus of the next N-protected amino acid to the amine group in the presence of a coupling reagent,
e) repeating steps c) and d) to form a thirty-four peptide sequence,
f) cleaving the peptide with a cocktail mixture from the resin to isolate a straight chain of crude Abaloparatide, and
g) purifying by reverse phase HPLC to isolate pure Abaloparatide.

2. The process according to claim 1, wherein the resin is selected from a Rink-Amide AM resin.

3. The process according to claim 1, wherein the coupling agent used for the coupling of the amino acids is selected from O-Benzotriazole-N,N,N′,N′-tetramethyl-uronium-hexaffuoro-phosphate (HBTU), 1 -Hydroxybenzotriazo (HOBt, ethyl cyano(hydroxyimino)acetate (Oxyma Pure) and Diisopropylethylamine (DIEA)

4. The process according to claim 1, wherein isolation of Abaloparatide is carried out by precipitating with an ether solvent selected from the group consisting of methyl tert-butyl ether, diethyl ether, t-butyl methyl ether, isopropyl ether and mixtures thereof.

5. The process according to claim 1, wherein the pure Abaloparatide has a purity of at least 99.0%.

6. The process according to claim 1, wherein the pure Abaloparatide contains low levels of impurities selected from des-Thr³³-Abaloparatide, (4-34)-Abaloparatide, (3-34)-Abaloparatide, des-Val²-Abaloparatide and Des-Ser³-Abaloparatide.

7. The process according to claim 1, wherein the pure Abaloparatide contains less than 0.10% Des-Thr³³-Abaloparatide.

8. The process according to claim 1, wherein the pure Abaloparatide contains less than 0.20% (4-34)-Abaloparatide.

9. The process according to claim 1, wherein the pure Abaloparatide contains less than 0.10% (3-34)-Abaloparatide.

10. The process according to claim 1, wherein the pure Abaloparatide contains less than 0.10% Des-Val²-Abaloparatide.

11. The process according to claim 1, wherein the pure Abaloparatide contains less than 0.10% Des-Ser³-Abaloparatide.

12. Abaloparatide containing as impurities, less than 0.10% Des-Thr³³-Abaloparatide, less than 0.20% (4-34)-Abaloparatide, less than 0.10% (3-34)-Abaloparatide, less than 0.10% Des-Val²-Abaloparatide and less than 0.10% Des-Ser³-Abaloparatide.
